(19) United States
(12) Patent Application Publication
(10) Pub. No.: US 2018/0361015 A1
(43) Pub. Date: Dec. 20, 2018

(54) Silk-Based Adhesives

(71) Applicant: Trustees of Tufts College, Medford, MA (US)

(72) Inventors: Kelly A. Burke, Grafton, MA (US); David L. Kaplan, Concord, MA (US)

(21) Appl. No.: 15/780,229

(22) PCT Filed: Nov. 29, 2016
(86) PCT No.: PCT/US2016/063932

§ 371 Date: May 30, 2018

Related U.S. Application Data
(60) Provisional application No. 62/261,120, filed on Nov. 30, 2015.

(51) Int. Cl. A61L 24/10; A61L 24/04; A61L 24/00

(52) U.S. Cl. CPC A61L 24/108; A61L 24/0042; A61L 24/046

(57) ABSTRACT

In some embodiments, the present invention provides compositions including silk fibroin, at least one hydrophilic agent, and at least one catechol donating agent, wherein the at least one hydrophilic agent and at least one catechol donating agent are conjugated to the silk fibroin. According to various embodiments, at least a portion of the silk fibroin may be crosslinked. In some embodiments, the silk fibroin is at least 50% (e.g., 60%, 70%, 80%, 90%, 95% or more) crosslinked. In some embodiments, the present invention also provides methods for making such compositions.

### BACKGROUND

The need for improved adhesives with particular properties is felt across a wide variety of industries. In particular, polymers that adhere to wet surfaces that are biocompatible, degradable, and devoid of animal-sourced materials are desired for applications as tissue adhesives. While a variety of adhesives have been developed, each has proved unsatisfactory for one reason or another including, for example, a propensity to significantly swell upon or shortly after application, poor water solubility, and/or poor degradation characteristics in vivo.

### SUMMARY OF THE INVENTION

The present invention provides new adhesive compositions that are biocompatible, biodegradable, and highly compatible with aqueous environments. In part, the present invention encompasses the surprising discovery that conjugation of catechol groups to modified forms of silk fibroin results in a strongly adhesive composition that is compatible with aqueous environments. An additionally surprising recognition encompassed by the present invention is that only low levels of hydrophilic agents are necessary to achieve solubilization and aqueous compatibility of provided compositions. In some embodiments, low levels are defined as 20 or fewer chains (e.g., 15, 10, 5, or less) associated per silk fibroin molecule. According to various embodiments, provided compositions are able to exhibit a high degree of resistance to swelling in aqueous environments which was unachievable with previously known biocompatible adhesives. In some embodiments, such swelling resistance may be due to crosslinking of at least a portion of the silk fibroin, for example, through beta sheet formation. Also in accordance with a variety of embodiments, provided compositions may also exhibit an unexpectedly high degree of adhesive strength, which may also be attributable, at least in part, to the formation of crosslinks in the silk fibroin.

In some embodiments, the present invention provides compositions including silk fibroin, at least one hydrophilic agent, and at least one catechol donating agent, wherein the at least one hydrophilic agent and at least one catechol donating agent are conjugated to the silk fibroin. According to various embodiments, at least a portion of the silk fibroin may be crosslinked. In some embodiments, the silk fibroin is at least 50% (e.g., 60%, 70%, 80%, 90%, 95% or more) crosslinked.

In some embodiments, the present invention also provides methods including the steps of providing a silk fibroin solution, associating the silk fibroin solution with at least one hydrophilic agent to form a solubilized silk fibroin solution, and conjugating the solubilized silk fibroin solution with at least one catechol donating agent to form an adhesive silk fibroin composition. In some embodiments, one or more of the intermediate products in provided methods may be lyophilized at or after one or more steps provided herein. In some embodiments, the silk fibroin solution is lyophilized prior to the associating step. In some embodiments, the silk fibroin solution is lyophilized prior to the conjugating step.

Various embodiments may include one or more hydrophilic agents. In some embodiments, a hydrophilic agent may be any hydrophilic molecule that may react with silk fibroin and increase the silk fibroin's solubility. One of skill will recognize that several parameters of a candidate hydrophilic molecule may be varied in order to tailor a particular embodiment. For example, molecular weight, charge (or lack thereof), and chain architecture (linear vs. branched, for example), may each be varied in order to design or select a hydrophilic agent for use in any particular embodiment. In some embodiments, the at least one hydrophilic agent is present in the composition in an amount at or below 10 substitutions (e.g., molecules, including small molecules or chains) per silk fibroin molecule. In some embodiments, the at least one hydrophilic agent is selected from the group consisting of poly(ethylene glycol), poly(glutamic acid), poly(lysine), glycosaminoglycans, sugars, and oligomers of sugars. In some embodiments, the at least one hydrophilic agent is present in the composition in an amount at or below 10 chains or molecules per silk fibroin molecule. In some embodiments, the at least one hydrophilic agent is present in the composition in an amount at or below 5 chains or molecules per silk fibroin molecule. In some embodiments, the at least one hydrophilic agent is poly(ethylene glycol) and is present in the composition in an amount at or below 10 poly(ethylene glycol) chains per silk fibroin molecule. In some embodiments, the at least one hydrophilic agent is poly(ethylene glycol) and is present in the composition in an amount at or below 5 poly(ethylene glycol) chains per silk fibroin molecule. In some embodiments, a glycosaminoglycan is selected from chitosan, heparin, heparin sulfate, chondroitin sulfate, keratin sulfurate, and/or hyaluronic acid.

In some embodiments, the present invention provides methods for selecting and/or characterizing appropriate hydrophilic agents for a particular application. In some embodiments, provided methods include assessing one or more of a candidate hydrophilic agent's molecular weight, flexibility of chains (persistence length) and charge to determine whether a particular hydrophilic agent is suitable for a particular use(s). In some embodiments, a desirable hydrophilic agent may be characterized as one which increases the solubility of silk fibroin in a particular condition or set of conditions, while not creating steric or electrostatic repulsion between the hydrophilic agent and the silk fibroin backbone.

According to various embodiments, any catechol-containing compound may serve as a catechol donating agent. In some embodiments, a catechol-containing compound is any compound which includes 6 membered aromatic carbon ring with OH substitutions in place of hydrogen at carbons 2 and 3 and includes a spacer with a reactive group on carbon 1 of the catechol ring. Any of a variety of reactive groups are compatible with various embodiments so long as they are capable of reacting with silk fibroin, for example, a reactive group may be or comprise a primary amine group. In some embodiments, the at least one catechol donating agent is selected from the group consisting of dopamine, norepinephrine, epinephrine, and L-3,4-dihydroxyphenylalanine. In some embodiments, the catechol donating agent (e.g., dopamine) is conjugated to the silk fibroin. In some embodiments, the conjugation is or comprises covalent bonding. In some embodiments, catechol donating agents attach a catechol to silk fibroin without oxidizing the hydroxyl groups on the catechol. In some embodiments, catechol donating agents deprotect the hydroxyl groups on a donated catechol after conjugation of the catechol to silk fibroin.

In some embodiments, the composition is characterized in that, upon exposure to an aqueous environment, the composition does not swell more than 50%, relative to its original size (e.g., not more than 45%, 40%, 35%, 30%, 25%). In some embodiments, the composition does not swell more than 20%, relative to its original size (e.g., not more than 15%, 10%, 5%, or less).

According to various embodiments, provided compositions may exhibit high degrees of adhesive strength. Adhesive strength may be characterized using any known physical parameter and method for measuring the same. In some embodiments, provided compositions are characterized as having an adhesive strength/force of at least about 20 kPa (e.g., at least about 25, 30, 40, 50, 60, 70, 80, 90, 100, 110, 120, 130, 140, 150, 200, 250, 300 kPa or more). In some embodiments, the composition is characterized as having an adhesive force of at least about 115 kPa. In some embodiments, the adhesive strength/force may vary considerably between the hydrated and non-hydrated states. In some embodiments, provided compositions in a hydrated state may be characterized as having an adhesive strength/force of at least 1 kPa (e.g., at least 2, 3, 4, 5, 10, 15, 20 kPa or more). In addition, in some embodiments, the adhesive strength/force may vary significantly depending on the surfaces used for adhesion, the concentration of the modified silk used in the reaction, and other environmental factors present. In some embodiments, increasing the roughness of a surface will increase the adhesive strength of a provided composition as compared to the same provided composition on a less rough surface. In some embodiments, increased concentrations of solubilized silk fibroin will result in increased adhesive strength.

In some embodiments, the silk fibroin is functionalized through carboxylation of at least a portion of the serine groups in the silk fibroin. In some embodiments, the silk fibroin in the silk fibroin solution is functionalized through carboxylation of at least a portion of the serine groups in the silk fibroin prior to the associating step.

In some embodiments, provided methods may include one or more additional processing or other steps. For example, in some embodiments, provided methods further comprise crosslinking at least a portion of the silk fibroin in the adhesive silk fibroin composition. In some embodiments, crosslinking occurs after the associating step. In some embodiments, crosslinking occurs after the conjugating step. While the crosslinking may occur via any applicable process, in some embodiments, the crosslinking of the silk fibroin includes at least one of sonication, vortexing, exposure to low pH environment, methanol treatment, exposure to water vapor, exposure to shear stress, exposure to salt, exposure to elevated pressure, addition of polyethylene glycol, and exposure to an electric field.

As used in this application, the terms “about” and “approximately” are used as equivalents. Any citations to publications, patents, or patent applications herein are incorporated by reference in their entirety. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWING

FIG. 1 shows an exemplary synthesis of dopamine-modified silk by (i) carboxylic acid enrichment; (ii) PEGylation of carboxylated silk; and (iii) dopamine functionalization. Reaction conditions (i): 1M ClCH₂COOH, 3M NaOH, 25° C., 30 min. NaH₂PO₄ (6 mg/ml), 25° C., 30 min. Neutralize, purify by dialysis. Reaction conditions (ii): Methoxy(poly(ethylene glycol)) activated with cyanuric chloride, 0.1 M Na₂B₄O₇ pH 9.5, 4° C., 2 hours. Purify by dialysis. Reaction conditions (iii): Dopamine hydrochloride, COMU, Et₃N, DMSO, 40° C., 16 hours. Purify by dialysis.

FIG. 2a-b ATR-FTIR spectra of products from PEGylation reactions on carboxylated silk fibroin: (a) “as cast” samples and (b) samples treated with methanol for 24 hours to induce beta sheet, where SF(no COOH) is silk fibroin without carboxyl modification and CarboxySF(x) is carboxylated silk fibroin with x PEG chains attached per molecule of fibroin. Vertical reference lines mark 1650, 1625, 1540, and 1515 cm⁻¹.

FIG. 3 ¹H NMR spectra in deuterated DMSO of purified products of dopamine reactions run on 10 minute boiled silk fibroin: (i) CarboxySF, (ii) CarboxySF-dopamine, (iii) dopamine hydrochloride, and (iv) COMU.

FIG. 4a-f Image of CarboxySF (a), CarboxySF(20) (b), CarboxySF(40) (c), CarboxySF-dopamine (d), CarboxySF(20)-dopamine (e), and CarboxySF(40)-dopamine (f) dissolved at 80 mg/mL in distilled water.

FIG. 5a-b Catechol quantification using Arnow's protocol. (a) UV-Vis spectra of CarboxySF(5) and CarboxySF(5)-dopamine. (b) Dopamine conjugated to the silk products, normalized to mole of product. + denotes significance (p<0.001) relative to CarboxySF(5) and CarboxySF(23) and ++ denotes significant difference at level p<0.005.

FIG. 6 Adhesion of dopamine-modified silks. Aluminum shims were adhered in a single lap configuration and pulled apart in tension. The average peak force to break the bonded shims is plotted with error bars denoting standard deviation. + denotes significance at p<0.001 and ++ denotes significance at p<0.02.

FIG. 7 Cell proliferation on dopamine modified silks, as assayed using Alamar Blue. Significant difference between labeled time point and previous time point is denoted by symbol, where + denotes p<0.001, $ denotes p<0.05, and # denotes not significant (p>0.05).

### DEFINITIONS

Agent: The term “agent” as used herein may refer to a compound or entity of any chemical class including, for example, polypeptides, nucleic acids, saccharides, lipids, small molecules, metals, or combinations thereof. As will be clear from context, in some embodiments, an agent can be or comprise a cell or organism, or a fraction, extract, or component thereof. In some embodiments, an agent is or comprises a natural product in that it is found in and/or is obtained from nature. In some embodiments, an agent is or comprises one or more entities that is man-made in that it is designed, engineered, and/or produced through action of the hand of man and/or is not found in nature. In some embodiments, an agent may be utilized in isolated or pure form; in some embodiments, an agent may be utilized in crude form. Some particular embodiments of agents that may be utilized in accordance with the present invention include small molecules, antibodies, antibody fragments, aptamers, nucleic acids (e.g., siRNAs, shRNAs, DNA/RNA hybrids, antisense oligonucleotides, ribozymes), peptides, peptide mimetics, etc. In some embodiments, an agent is or comprises a polymer. In some embodiments, an agent is not a polymer and/or is substantially free of any polymer. In some embodiments, an agent contains at least one polymeric moiety.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated: Two events or entities are “associated” with one another, as that term is used herein, if the presence, level and/or form of one is correlated with that of the other. In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Biocompatible: The term “biocompatible”, as used herein, refers to materials that do not cause significant harm to living tissue when placed in contact with such tissue, e.g., in vivo. In certain embodiments, materials are “biocompatible” if they are not toxic to cells. In certain embodiments, materials are “biocompatible” if their addition to cells in vitro results in less than or equal to 20% cell death, and/or their administration in vivo does not induce significant inflammation or other such adverse effects.

Biodegradable: As used herein, the term “biodegradable” refers to materials that, when introduced into cells, are broken down (e.g., by cellular machinery, such as by enzymatic degradation, by hydrolysis, and/or by combinations thereof) into components that cells can either reuse or dispose of without significant toxic effects on the cells. In certain embodiments, components generated by breakdown of a biodegradable material are biocompatible and therefore do not induce significant inflammation and/or other adverse effects in vivo. In some embodiments, biodegradable polymer materials break down into their component monomers. Exemplary biodegradable polymers include, for example, polymers of hydroxy acids such as lactic acid and glycolic acid, including but not limited to poly(hydroxyl acids), poly(lactic acid)(PLA), poly(glycolic acid)(PGA), poly(lactic-co-glycolic acid)(PLGA), and copolymers with PEG, polyanhydrides, poly(ortho)esters, polyesters, polyurethanes, poly(butyric acid), poly(valeric acid), poly(caprolactone), poly(hydroxyalkanoates, poly(lactide-co-caprolactone), blends and copolymers thereof. Many naturally occurring polymers are also biodegradable, including, for example, proteins such as albumin, collagen, gelatin and prolamines, for example, zein, and polysaccharides such as alginate, cellulose derivatives and polyhydroxyalkanoates, for example, polyhydroxybutyrate blends and copolymers thereof. Those of ordinary skill in the art will appreciate or be able to determine when such polymers are biocompatible and/or biodegradable derivatives thereof (e.g., related to a parent polymer by substantially identical structure that differs only in substitution or addition of particular chemical groups as is known in the art).

Catechol: As used herein, the term “catechol” refers to an organic compound with the molecular formula C₆H₄(OH)₂, and is sometimes referred to as 1,2-dihydrobenzene. For reference, an exemplary chemical structure of a catechol group is shown below:

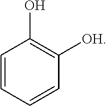

Fibroin: As used herein, the term “fibroin” includes silkworm fibroin and/or insect or spider silk protein. Lucas et al., 13 Adv. Protein Chem. 107-242 (1958). For example, silk fibroin may be obtained from a solution containing a dissolved silkworm silk or spider silk. The silkworm silk protein is obtained, for example, from B. mori, and the spider silk is obtained from Nephila clavipes. Alternatively, silk proteins suitable for use in the present invention can be obtained from a solution containing a genetically engineered silk, such as from bacteria, yeast, mammalian cells, transgenic animals or transgenic plants. See, e.g., WO 97/08315; U.S. Pat. No. 5,245,012.

Hydrophilic: As used herein, the term “hydrophilic” and/or “polar” refers to a tendency to mix with, or dissolve easily in, water.

Hydrophobic: As used herein, the term “hydrophobic” and/or “non-polar”, refers to a tendency to repel, not combine with, or an inability to dissolve easily in, water.

Reference: as used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Substantially: As used herein, the term “substantially” refers to the qualitative condition of exhibiting total or near-total extent or degree of a characteristic or property of interest. One of ordinary skill in the biological or chemical arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

### DETAILED DESCRIPTION

The present invention provides, inter alia, adhesive compositions that are biocompatible, biodegradable, highly tunable, compatible with aqueous environments, and exhibit surprisingly high levels of adhesive strength and resistance to swelling upon exposure to an aqueous environment as well as methods for making and using such compositions. In some embodiments, provided compositions include one or more therapeutic agents.

In some embodiments, the present invention provides compositions including silk fibroin, at least one hydrophilic agent, and at least one catechol donating agent, wherein the at least one hydrophilic agent and at least one catechol donating agent are conjugated to the silk fibroin. According to various embodiments, at least a portion of the silk fibroin may be crosslinked. In some embodiments, the silk fibroin is at least 50% (e.g., 60%, 70%, 80%, 90%, 95% or more) crosslinked.

Silk

Silks are protein-based biopolymers produced by many insects and arachnids. Domesticated silkworms, such as Bombyx mori, are used to produce silk for the textile industry. Cocoons fabricated by the B. mori silkworm are comprised of two main protein types: sericins and fibroin. Fibroin consists of a heavy chain (˜390 kDa) and a light chain (˜26 kDa) present in a 1:1 ratio and linked by a disulfide bond. Sericins are a family of gluelike proteins ranging from 20 kDa to 310 kDa that coat the fibroin chains. Purified (removal of sericin) silkworm fibers, as well as purified regenerated fibroin, as a biomaterial, has provided an emerging biomaterial platform for many needs in medical devices, tissue engineering, and tissue regeneration.

Silk fibroin has an amino acid sequence consisting of repeats of glycine-alanine-glycine-alanine-glycine-serine (GAGAGS) that self-assemble to form beta sheets. Formation of beta sheets may be triggered by various processing techniques that involve energy input (e.g., sonication or vortexing), lowering the solution pH, or through dehydration of the protein by removal of water (e.g., by methanol treatment). Beta sheets are highly crystalline and serve to physically crosslink the protein through intra- and inter-molecular hydrogen bonding and van der Waals interactions. The beta sheet imparts impressive mechanical properties to the fibroin and renders the material insoluble in water. In some embodiments, beta sheet content may also affect the mechanical properties of certain materials generated from silk fibroin, including swelling and degradation. In some embodiments, degradation may be complete and the rate can be tuned to be longer in vivo than other commonly studied protein polymers for biomaterials (e.g., collagens and elastins).

Silk fibroin is a unique biopolymer that can be reconfigured from its native or synthesized states in various shapes and conformations, and has been used in biomedical applications for many years. Applications range from suture materials to tissue scaffolds used in the development of engineered tissues in the body, such as tendons, cartilage and ligaments. The forms of the silk required for particular applications vary. As such, significant research has been devoted to the development of silk films (Jin et al., 15 Adv. Funct. Matter 1241-47 (2005)), non-woven mats (Jin et al., 25 Biomats. 1039-47 (2004)), sponges (porous scaffolds) (Karageorgiou et al., Part A J. Biomed. Mats. Res. 324-34 (2006)), gels (Wang et al, 29 Biomats. 1054-64 (2008)), and other forms (Sofia et al., 54 J. Biomed. Materials Res. 139-48 (2000)).

For each of these forms, insect-derived silk is typically processed into solution using a two-stage procedure. In the case of silkworm silk, cocoons from Bombyx mori silkworms are boiled in an aqueous solution and subsequently rinsed to remove the glue-like sericin protein that covers the natural silk. The extracted silk fibroin is then solubilized (i.e., dissolved) in LiBr before being dialyzed in water. The solubilized silk fibroin concentration can then be adjusted according to the intended use. See U.S. Patent Application Pub. No. 20070187862. Alternatively, recombinant silk proteins may be used. These have proved advantageous when using spider silk because arachnid-derived silk proteins are often more difficult to collect in quantity. Kluge et al., 26(5) Trends Biotechnol. 244-51 (2008). Moreover, recombinant silk fibroin may be engineered to express heterologous proteins or peptides, such as dentin matrix protein 1 and RGD, providing additional biofunctionality to the silk fibroin proteins. Huang et al., 28(14) Biomats. 2358-67 (2007); Bini et al., 7(11) Biomacromolecules 3139-45 (2006).

According to various embodiments, the silk protein suitable for use in the present invention is preferably fibroin or related proteins (i.e., silks from spiders). Preferably, fibroin or related proteins are obtained from a solution containing a dissolved silkworm silk or spider silk. The silkworm silk is obtained, for example, from Bombyx mori. Spider silk may be obtained from Nephila clavipes. In the alternative, the silk protein suitable for use in the present invention can be obtained from a solution containing a genetically engineered silk, such as from bacteria, yeast, mammalian cells, transgenic animals or transgenic plants. See, for example, WO 97/08315 and U.S. Pat. No. 5,245,012.

Preparation of silk fibroin solutions has been described previously, e.g., in WO 2007/016524, which is incorporated herein by reference in its entirety. The reference describes not only the preparation of aqueous silk fibroin solutions, but also such solutions in conjunction with bioactive agents. By way of example, a silk fibroin solution can be prepared by any conventional method known to one skilled in the art. For example, B. mori cocoons are boiled for about 30 minutes in an aqueous solution. Preferably, the aqueous solution is about 0.02M Na₂CO₃. The cocoons are rinsed, for example, with water to extract the sericin proteins and the extracted silk is dissolved in an aqueous salt solution. Salts useful for this purpose include, lithium bromide, lithium thiocyanate, calcium nitrate or other chemical capable of solubilizing silk. A strong acid such as formic or hydrochloric may also be used. Preferably, the extracted silk is dissolved in about 9-12 M LiBr solution. The salt is consequently removed using, for example, dialysis. According to various embodiments, boiling time may vary from approximately 5 to 10 minutes of boiling to 60 minutes of boiling or more, depending upon the size(s) of silk fibroin fragments desired for a particular embodiment.

In some embodiments, a silk solution may be concentrated using, for example, dialysis against a hygroscopic polymer, for example, PEG, a polyethylene oxide, amylose or sericin. In some embodiments, PEG is of a molecular weight of 8,000-10,000 g/mol and has a concentration of 25-50%. In some embodiments, any dialysis system can be used. In some embodiments, dialysis may be for a time period sufficient to result in a final concentration of aqueous silk solution between 10-30%, for example, dialysis for 2-12 hours.

In some embodiments, biocompatible polymers can also be added to a silk solution to generate composite matrices in the methods and processes of the present invention. Exemplary biocompatible polymers useful in the present invention include, for example, polyethylene oxide (PEO) (U.S. Pat. No. 6,302,848), polyethylene glycol (PEG) (U.S. Pat. No. 6,395,734), collagen (U.S. Pat. No. 6,127,143), fibronectin (U.S. Pat. No. 5,263,992), keratin (U.S. Pat. No. 6,379,690), polyaspartic acid (U.S. Pat. No. 5,015,476), polylysine (U.S. Pat. No. 4,806,355), alginate (U.S. Pat. No. 6,372,244), chitosan (U.S. Pat. No. 6,310,188), chitin (U.S. Pat. No. 5,093,489), hyaluronic acid (U.S. Pat. No. 387,413), pectin (U.S. Pat. No. 6,325,810), polycaprolactone (U.S. Pat. No. 6,337,198), polylactic acid (U.S. Pat. No. 6,267,776), polyglycolic acid (U.S. Pat. No. 5,576,881), polyhydroxyalkanoates (U.S. Pat. No. 6,245,537), dextrans (U.S. Pat. No. 5,902,800), and polyanhydrides (U.S. Pat. No. 5,270,419). In some embodiments, two or more biocompatible polymers can be used.

In accordance with various embodiments, a silk solution may comprise any of a variety of concentrations of silk fibroin. In some embodiments, a silk solution may comprise 0.1 to 30% by weight silk fibroin. In some embodiments, a silk solution may comprise between about 0.5% and 30% (e.g., 0.5% to 25%, 0.5% to 20%, 0.5% to 15%, 0.5% to 10%, 0.5% to 5%, 0.5% to 1.0%) by weight silk fibroin, inclusive. In some embodiments, a silk solution may comprise at least 0.1% (e.g., at least 0.5%, 1%, 2%, 3%, 4%, 5%, 6%, 7%, 8%, 9%, 10%, 15%, 20%, 25%) by weight silk fibroin. In some embodiments, a silk solution may comprise at most 30% (e.g., at most 25%, 20%, 15%, 14%, 13%, 12% 11%, 10%, 5%, 4%, 3%, 2%, 1%) by weight silk fibroin.

Silk fibroin solutions used in methods and compositions described herein may be obtained from a solution containing a dissolved silkworm silk, such as, for example, from Bombyx mori. Alternatively, a silk fibroin solution is obtained from a solution containing a dissolved spider silk, such as, for example, from Nephila clavipes. Silk fibroin solutions can also be obtained from a solution containing a genetically engineered silk. Genetically engineered silk can, for example, comprise a therapeutic agent, e.g., a fusion protein with a cytokine, an enzyme, or any number of hormones or peptide-based drugs, antimicrobials and related substrates.

Provided silk compositions described herein, and methods of making and/or using them may be performed in the absence of any organic solvent. Thus, in some embodiments, provided compositions and methods are particularly amenable to the incorporation of labile molecules, such as bioactive agents or therapeutics, and can, in certain embodiments, be used to produce controlled release biomaterials. In some embodiments, such methods are performed in water only.

Hydrophilic Agents

Various embodiments may include one or more hydrophilic agents. In some embodiments, a hydrophilic agent may be any hydrophilic molecule that may react with silk fibroin and increase the silk fibroin's solubility. One of skill will recognize that several parameters of a candidate hydrophilic molecule may be varied in order to tailor a particular embodiment. For example, molecular weight, charge (or lack thereof), and chain architecture (linear vs. branched, for example), may each be varied in order to design or select a hydrophilic agent for use in any particular embodiment.

According to various embodiments, it may be advantageous to tune the degree of hydrophilicity and/or solubility to a particular application. Accordingly, it may be advantageous in some embodiments to include a relatively low concentration of hydrophilic agents in a particular composition, as compared to the theoretical loading capacity of the silk fibroin. Without wishing to be held to a particular theory, it is possible that in some embodiments, keeping the concentration of the hydrophilic agent(s) low will reduce the effects of potential steric interference with the catechol donating agent(s), therapeutic agent(s), or other components of particular provided compositions. By way of example, in some embodiments, the at least one hydrophilic agent is present in the composition in an amount at or below 10 substitutions (e.g., molecules, including small molecules or chains) per silk fibroin molecule. By way of more specific example, wherein the at least one hydrophilic agent is or comprises poly(ethylene glycol), also referred to herein as PEG, in some embodiments, the at least one hydrophilic agent is poly(ethylene glycol) and is present in the composition in an amount at or below 10 poly(ethylene glycol) chains per silk fibroin molecule. In some embodiments, the at least one hydrophilic agent is poly(ethylene glycol) and is present in the composition in an amount at or below 5 poly(ethylene glycol) chains per silk fibroin molecule.

In accordance with several embodiments, any application appropriate hydrophilic agent(s) may be used. In some embodiments, the at least one hydrophilic agent is selected from the group consisting of poly(ethylene glycol), poly(glutamic acid), poly(lysine), glycosaminoglycans, sugars, and sugar oligomers. In some embodiments, a glycosaminoglycan is selected from chitosan, heparin, heparin sulfate, chondroitin sulfate, keratin sulfurate, and/or hyaluronic acid.

Poly(ethylene glycol)/PEG

In some embodiments, the at least one hydrophilic agent will be or comprise poly(ethylene glycol), or PEG. In some embodiments, the PEG may be in any of a branched, star, or comb form.

In some embodiments, a PEG may be a multi-arm PEG derivative (e.g., 2-arm, 4-arm, 8-arm, and 12-arm, etc.). In some embodiments, provided compositions may include two or more PEG components. In some embodiments, each of the PEG components can be a multi-arm PEG derivative (e.g., 2-arm, 4-arm, 8-arm, and 12-arm, etc.). The term “multi-arm PEG derivatives” described herein refers to a branched poly(ethylene glycol) with at least about 2, at least about 4, at least about 6, at least about 8, at least about 12 PEG polymer chains or derivatives thereof (“arms”) or more. Multi-arm or branched PEG derivatives include, but are not limited to, forked PEG and pendant PEG. An example of a forked PEG can be represented by PEG-YCHZ₂, where Y is a linking group and Z is an activated terminal group linked to CH by a chain of atoms of defined length. The International Patent Application No. WO 99/45964, the content of which is incorporated herein by reference in its entirety, discloses various forked PEG structures that can be used for some embodiments of the present invention. For example, the chain of atoms linking the Z functional groups to the branching carbon atom can serve as a tethering group and can comprise, for example, alkyl chains, ether chains, ester chains, amide chains and combinations thereof. A pendant PEG can have functional groups, such as carboxyl, covalently attached along the length of the PEG segment rather than at the end of the PEG chain. The pendant reactive groups can be attached to the PEG segment directly or through a linking moiety, such as alkene.

In various embodiments, the molecular weight of each of the PEG components or other synthetic polymers can independently vary depending on the desired application. In some embodiments, the molecular weight (MW) is about 100 Da to about 100,000 Da, about 1,000 Da to about 20,000 Da, or about 5,000 Da to about 15,000 Da. In some embodiments, the molecular weight of the PEG components is about 10,000 Da.

Catechol Donating Agents

According to various embodiments, any catechol-containing compound may serve as a catechol donating agent. In some embodiments, a catechol-containing compound is any compound which includes 6 membered aromatic carbon ring with OH substitutions in place of hydrogen at carbons 2 and 3 and includes a spacer with a reactive group on carbon 1 of the catechol ring. Any of a variety of reactive groups are compatible with various embodiments so long as they are capable of reacting with silk fibroin, for example, a reactive group may be or comprise a primary amine group.

In some embodiments, the at least one catechol donating agent is selected from the group consisting of dopamine, norepinephrine, epinephrine, 3,4-dihydroxy-9,10-seco-androst-1,3,5(10)-triene-9,17-dione (DHSA), 3,4-dihydroxystyrene, L-3,4-dihydroxyphenylalanine, and combinations thereof. In some embodiments, a catechol donating agent is an analog or derivative of one of the specific agents listed above.

Without wishing to be held to a particular theory, it is possible that conjugation of catechol groups to silk fibroin allows for the exploitation of catechol groups to associate with a wide variety of materials while being enhanced through association with the silk fibroin and allowing for improved properties of the composition as a whole, beyond what each individual component would be capable of achieving.

Methods of Making and Processing

In some embodiments, the present invention also provides methods including the steps of providing a silk fibroin solution (e.g., a degummed silk fibroin solution), associating the silk fibroin solution with at least one hydrophilic agent to form a solubilized silk fibroin solution, and conjugating the solubilized silk fibroin solution with at least one catechol donating agent to form an adhesive silk fibroin composition. Certain exemplary methods are described in the Examples below.

Associating/Conjugating

In some embodiments, the hydrophilic agent and/or catechol donating agent (e.g., dopamine) are conjugated to the silk fibroin. As used herein, the term “conjugation” or “conjugated” refers to any manner of physically associating a silk fibroin with a catechol group that is stable before and/or after catechol oxidation. In some embodiments, the conjugation is or comprises covalent bonding. In some embodiments, the mode of conjugation may be or comprise UV light and/or metal ion reactions.

Any application-appropriate method(s) may be used to conjugate the hydrophilic agent(s) and/or catechol donating agent(s) to the silk fibroin. Exemplary methods include Williamson ether synthesis, carbodiimide coupling chemistry, cyanuric chloride-mediated coupling, Fischer esterification, Grignard reaction, diazonium coupling chemistry, and enzyme-mediated coupling.

Lyophilization

In some embodiments, one or more of the intermediate products in provided methods may be lyophilized at or after one or more steps provided herein. Potential advantages of lyophilizing certain intermediates include enhancing ease of storage or transport. Any known method of lyophilizing the materials described herein may be used in accordance with certain embodiments such as freeze drying or desiccation. By way of example, in some embodiments, the silk fibroin solution is lyophilized prior to the associating step. By way of further example, in some embodiments, the silk fibroin solution is lyophilized prior to the conjugating step.

Exemplary, non-limiting methods for lyophilizing provided compositions include freeze-drying, spray drying, and/or vacuum concentration.

Crosslinking

In some embodiments, provided methods further comprise crosslinking at least a portion of the silk fibroin in the adhesive silk fibroin composition. As used herein, the term “crosslinking” is synonymous with inducing beta sheet formation. In some embodiments, crosslinking occurs after the associating step. In some embodiments, crosslinking occurs after the conjugating step. While the crosslinking may occur via any applicable process, in some embodiments, the crosslinking of the silk fibroin includes at least one of sonication, vortexing, exposure to low pH environment, methanol treatment, exposure to water vapor, exposure to shear stress, exposure to salt, exposure to elevated pressure, addition of polyethylene glycol, and exposure to an electric field. Those of skill in the art will understand several processes for inducing crosslinks in provided compositions including those described, inter alia, in Wang, Y. Y.; Cheng, Y. D.; Liu, Y.; Zhao, H. J.; Li, M. Z. Advanced Materials Research 2011, 175-176, 143-148; Samal, S. K.; Kaplan, D. L.; Chiellini, E. Macromolecular Materials and Engineering 2013, 298, 1201-1208; Liu, G.; Xiong, S.; You, R.; Wang, L.; Li, M. Advanced Materials Research 2013, 634-638, 1165-1169; Yucel, T.; Cebe, P.; Kaplan, D. L. Biophys. J. 2009, 97, 2044-2050; and Jin, H. J.; Park, J.; Karageorgiou, V.; Kim, U. J.; Valluzzi, R.; Cebe, P.; Kaplan, D. L. Advanced Functional Materials 2005, 15, 1241-1247, among others.

In some embodiments, provided compositions are at least 1% cross-linked (e.g., at least 1% of the silk fibroin present in a composition is in beta-sheet form and/or otherwise cross-linked). In some embodiments, provided compositions are at least 10% cross-linked (e.g., at least 10% of the silk fibroin present in a composition is in beta-sheet form and/or otherwise cross-linked). In some embodiments, provided compositions are more than 1% cross-linked (e.g., at least 2%, 3%, 4%, 5%, 6%, 7%, 8%, 9%, 10%, 15%, 20%, 25%, 30%, 40%, 50%, 60%, 70%, 80%, 90%, or more). In some embodiments, provided compositions comprise substantially no silk fibroin in beta-sheet form.

Therapeutic Agents

In some embodiments, provided compositions may include one or more therapeutic agents. In some embodiments, to form these compositions, a silk solution is mixed with a therapeutic agent prior to forming the composition or loaded on or in the composition after it is formed.

The variety of different therapeutic agents that can be used in conjunction with the biomaterials of the present invention is vast and includes small molecules, proteins, peptides and nucleic acids. In general, therapeutic agents which may be administered in accordance with various embodiments of the present invention include, without limitation: anti-infectives such as antibiotics and/or antiviral agents; chemotherapeutic agents (i.e., anticancer agents); anti-rejection agents; analgesics and analgesic combinations; anti-inflammatory agents; hormones such as steroids; growth factors, bone morphogenic-like proteins (i.e., GFD-5, GFD-7 and GFD-8), epidermal growth factor (EGF), fibroblast growth factor (i.e., FGF 1-9), platelet derived growth factor (PDGF), insulin like growth factor (e.g., IGF-I and IGF-II), transforming growth factors (i.e., TGF-β-III), vascular endothelial growth factor (VEGF); anti-angiogenic proteins such as endostatin, and other naturally derived or genetically engineered proteins, polysaccharides, glycoproteins, or lipoproteins. Exemplary growth factors are described in The Cellular and Molecular Basis of Bone Formation and Repair by Vicki Rosen and R. Scott Thies, published by R. G. Landes Company, the disclosure of which is hereby incorporated herein by reference.

Additionally, in some embodiments, provided compositions may be used to deliver a wide variety of therapeutic agents, including, but not limited to, pharmacological materials, vitamins, sedatives, steroids, hypnotics, antibiotics, chemotherapeutic agents, prostaglandins, and radiopharmaceuticals. In accordance with several embodiments, provided compositions may be suitable for delivery of the above materials and others including but not limited to proteins, peptides, nucleotides, carbohydrates, simple sugars, cells, genes, anti-thrombotics, anti-metabolics, growth factor inhibitor, growth promoters, anticoagulants, antimitotics, fibrinolytics, anti-inflammatory steroids, and monoclonal antibodies.

Provided compositions including one or more therapeutic agents may be formulated in a variety of ways, for example, by mixing one or more therapeutic agents with the polymer used to make the material. Alternatively, a therapeutic agent could be coated on to the material preferably with a pharmaceutically acceptable carrier. Any pharmaceutical carrier can be used that does not dissolve the silk material. In some embodiments, therapeutic agents may be present as a liquid, a finely divided solid, or any other appropriate physical form. In some embodiments, one or more therapeutic agents may be partially or substantially completely encapsulated within a provided composition.

Uses

Compositions and methods provided herein may be used for a wide variety of applications. In some embodiments, provided methods and compositions are useful for producing tissue adhesives and or sealants for use in surgical or other wound healing scenarios. In some embodiments, provided compositions may include one or more therapeutic agents that enhance patient outcomes and/or recovery. In some embodiments, provided compositions are able to provide adhesion during wound healing and also allow for cell and/or tissue ingrowth in order to facilitate complete recovery. According to various embodiments, provided compositions are compatible with cell growth and/or cell survival. In some embodiments, provided compositions may be or comprise surface coatings and may also provide barrier layers for an underlying material, lubricity for the surfaces, and/or fouling control.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### EXAMPLES

The following Example describes the creation of exemplary embodiments of the present invention including both compositions and methods for making the same. Provided compositions enjoy enhanced properties over previously known adhesives including, but not limited to compatibility with aqueous environments, biocompatibility, biodegradability, enhanced adhesive strength particularly in aqueous environments, and resistance to swelling upon exposure to an aqueous environment.

Unless otherwise specified the material and methods used in the following Examples are as described below:

Instruments

Instruments. ¹H NMR was performed using an Advance III NMR (Bruker BioSpin, Billerica Mass., USA) operating at 500 MHz and equipped with Topspin Software (version 2.1 Bruker). Fourier transform infrared spectroscopy was performed using a FT/IR-6200 Spectrometer (Jasco, Japan) that was equipped with a triglycine sulfate detector and run in attenuated total reflection (ATR) mode. Fourier Self-Deconvolution (FSD) of the infrared spectra covering the Amide I region (1595˜1705 cm⁻¹) was performed with Opus 5.0 software (Bruker Optics Corp., Billerica Mass., USA). Quantification of catechol conjugation and cell proliferation on the modified silks was achieved using chemical assays coupled with absorbance and fluorescence measurements obtained using a SpectraMax M2 plate reader (Molecular Devices, Sunnyvale, Calif., USA). Adhesion tests were performed on an Instron 3366 testing frame equipped with a 100 N load cell (Norwood, Mass., USA).

Reagents

Reagents. Dopamine hydrochloride (puriss, >98.5%), COMU (97%), anhydrous trimethylamine (Et₃N, 99%), anhydrous dimethyl sulfoxide (DMSO), sodium carbonate (>99.5%), lithium bromide (>99%), sodium phosphate monobasic (>99%), chloroacetic acid (>99%), methoxypolyethylene glycol activated with cyanuric chloride (average Mw 5000 g/mol), sodium periodate (>99.8%), sodium molybdate (98%), sodium nitrite (>97%), deuterated dimethyl sulfoxide-D6 (d-DMSO, 99.96%), and deuterium oxide (D₂O, 99.99%) were obtained from Aldrich Chemical Company (St. Louis, Mo., USA). Hydrochloric acid, sodium hydroxide, and regenerated cellulose dialysis membranes (MWCO 3500, 6000-8000, or 12000-14000 g/mol) were purchased from Fisher Scientific. Deionized water (DI) at 18 MOhm was obtained from an in-house purification system.

Purification of Silk Fibroin

Removal of sericins from B. mori silk fibroin was achieved by a previously published procedure, with slight modifications as noted. In brief, 5 g of cut cocoons were added to 2 L of 0.2 M NaHCO₃ solution at 100° C. The cocoons were continuously rotated and separated during a 10 min boiling time, which was shorter than the referenced procedure (30 min boiling time) to afford higher molecular weight fibroin. After 10 min, the degummed fibers were placed in 2 L of distilled water and rinsed for 20 min. This rinsing procedure was repeated two additional times to remove sericins (maximum content 10wt-%) from the fibroin, after which the fibers were dried overnight at room temperature. The yield of the dried degummed fibers was about 3.7 g or 74% on average.

To dissolve the silk fibroin, 1 g of degummed fibers was added to 5 mL of a 9.3 M LiBr solution. Compared to previous reports that use 4 mL of 9.3 M LiBr solution to dissolve silk boiled for 30 min, the concentration of silk fibers in LiBr solution was lower here because it was found that the silk fibroin boiled for 10 min was slightly more difficult to dissolve than the 30 min boiled fibroin. Thus, the volume of LiBr solution was increased in this work. Removal of salts proceeded as previously published, with the solution placed in 3500 MWCO dialysis tubing and dialyzed against 2 L of DI water per 12 mL of silk solution. The water was changed at regular intervals (1, 3, 6, 18, 30, 42 hours) before removal from tubing and centrifugation (8700 RPM, 4° C., 20 min). Settled particulate was removed by pouring off the silk solution into a new container, and the silk solution was centrifuged again. The clarified silk fibroin solution was then transferred to a new container and the concentration of fibroin was determined by weighing a known volume of silk that was dried overnight at 55° C. Typical concentration of the silk fibroin solution was 55 mg/mL.

Enrichment of Silk Fibroin

Enrichment of carboxyl groups on fibroin. To increase reactive groups for the dopamine reaction, the hydroxyl groups of serine were converted to carboxylic acid groups using an established procedure. In brief, the purified silk fibroin solution was added to a round bottom flask and diluted to 45 mg/mL using distilled water. Next, 10 M NaOH was added to the flask to yield a final concentration of 3 M NaOH. This solution was stirred for 2-3 min before chloroacetic acid was added to give a final concentration of 1 M. This solution was stirred at room temperature for 1 hour before stopping with the addition of NaH₂PO₄ (6.9 mg/mL). The reaction was then placed on ice and neutralized to pH 7 by dropwise addition of 12 M HCl. The use of the ice bath prevented the silk solution from large temperature increases during titration. The solution was allowed to stir for 30 min before transferring to dialysis tubing and dialyzing against distilled water. Like the dialysis described above, the solution placed in 3500 MWCO dialysis tubing and dialyzed against DI water, which was changed at regular intervals (1, 3, 6, 18, 30, 42 hours). The product of this reaction is named CarboxySF.

Because the concentration of CarboxySF post dialysis was very low, the dialysis tubing containing the silk solution was placed on a clean surface in a chemical fume hood and allowed to concentrate by removal of water for 48 hours. Two alternatives for this concentration method were considered, including lyophilization followed by dissolution at a higher concentration or concentrating by dialyzing against a solution containing a high molecular weight poly(ethylene glycol) (PEG), which will draw water out of the tubing yet not permit PEG to enter the tubing. The use of the fume hood air flow was selected over the lyophilization/dissolution route because lyophilization can induce beta sheet formation in the silk, rendering it more difficult to resolubilize in water. Concentration by air flow was selected instead of dialyzing against a PEG solution to avoid analytical complications, as some of these derivatives will be conjugated with PEG in downstream reactions. The typical concentration of CarboxySF after this concentration step was 15 mg/mL.

Conjugation of Poly(ethylene glycol) to CarboxySF

Poly(ethylene glycol) (PEG) was conjugated to tyrosine to increase the hydrophilicity of CarboxySF. Different amounts of PEG were conjugated to CarboxySF using the same procedure. Materials are named as CarboxySF(x), where x is the number of PEG chains conjugated per molecule of silk. As an example, the synthesis of CarboxySF(29) is described, where the following synthesis led to 6 PEG chains conjugated per molecule of silk fibroin. The degree of conjugation was quantified by ¹H NMR, as described later. To synthesize CarboxySF(29), 395 mL of an aqueous solution of CarboxySF (11.4 mg/mL) was added to a round bottom flask. Commercially available sodium borate buffer mixture (BupH Borate Buffer, Fisher) was dissolved in the silk solution to give 0.1 M borate buffer, and the pH was adjusted to 9.5 using 10 M NaOH. The solution was cooled to 4° C. before adding 3.31 g of cyanuric chloride activated methoxypoly(ethylene glycol) (Aldrich, average M_{w} 5,000 g/mol). The reaction was allowed to proceed for 2 hours with stirring at 4° C. To conjugate different amounts of PEG to the CarboxySF, the same conditions were employed but the PEG content was varied. Because the PEG reaction was found to reproducibly lead to 34% conjugation efficiency, the amount of PEG added to each reaction was 2.94 times larger than the theoretical amount needed to achieve the desired amount of PEG conjugation, and this was found to lead to highly reproducible reactions. A control reaction was prepared in the same manner, except that no cyanuric chloride activated methoxypoly(ethylene glycol) was added to this reaction, to confirm that the reaction workup did not change the silk. All reactions were worked up by dialysis in 12,000-14,000 MWCO tubing. For the first two changes, the dialysis was conducted against phosphate buffered saline at 4° C., and for the last 3 dialysis steps the dialysis was run against distilled water at 4° C.

Lyophilization of CarboxySF Solutions

Aqueous solutions of CarboxySF or CarboxySF(x) were placed in 50 mL polypropylene tubes and placed in a dry ice/isopropanol slurry for 2 hours to freeze the solutions. The samples were then transferred to a lyophilizer, where they were dried for 4 days.

Conjugation of Dopamine to Silk Fibroin

Lyophilized CarboxySF or CarboxySF(x) were dissolved in anhydrous dimethyl sulfoxide (DMSO, Aldrich) in a round bottom flask at room temperature and at a concentration of 67 mg/mL and purged with argon. No stirring was applied during the dissolution process, which took several hours to reach a homogenous solution. Dopamine hydrochloride (Aldrich) was then added to the solution at a 10-fold molar excess relative to carboxylic acid groups on the modified silk. For example, for 10 g of CarboxySF, 25.1 g dopamine hydrochloride was added to the solution, but for 10 g of CarboxySF(23), 21.1 g dopamine hydrochloride was added because of the PEG chain contribution to the modified silk's molecular weight. Next, COMU (Aldrich) was dissolved in the solution at a 2 fold molar excess relative to carboxylic acid functional groups. The reaction vessel was once again purged with argon gas, and the solution was subjected to gentle heating by placement in a 40° C. water bath to facilitate complete dissolution of the reactants. Once a homogenous solution was achieved, an 8 fold molar excess of triethylamine (Aldrich) was added to the solution. Once again, the vessel was purged with argon before being placed in a 40° C. incubator with gentle shaking (50 RPM). The reaction proceeded in this manner for 16 hours, before the reacting mixture was removed and transferred to dialysis tubing (6,000-8,000 MWCO). The reaction was dialyzed against degassed DMSO in a sealed container. Upon completion of the dialysis, which was judged by the absence of a change in the color of the solvent that typically occurred after 48 hours of dialysis, the solution was transferred to 50 mL polypropylene tubes and frozen using a dry ice/isopropanol slurry for 2 hours. The frozen samples were then transferred to a lyophilizer where they were dried for 5 days. A second dialysis was performed after lyophilization, where the catechol-functionalized silks were placed in dialysis tubing (20,000 MWCO) and dialyzed against 8 M urea (pH 6.1) for 2 hours, phosphate buffered saline (pH 6.1) 2 hours, phosphate buffered saline for 2 hours (pH 6.1), and finally distilled water for 48 h (pH 6.0). The conditions for this second dialysis, which is referred to as a washing protocol or an extraction for clarity, was guided by a separate experiment, as described below.

To ensure that dopamine was chemically coupled to silk fibroin and not absorbed to the silk, a washing protocol was developed. CarboxySF and dopamine were dissolved in water at the same concentration that was used in the catechol coupling reaction (50 mg/mL CarboxySF, 125 mg/mL dopamine). The mixture was then placed in dialysis tubing (20,000 MWCO) and dialyzed against one of the following solutions, which were all adjusted to pH 5.9-6.1 and degassed with argon to reduce catechol oxidation. Each of the washing protocols consisted of three solutions that the solution was dialyzed against for 2 hours. For Protocol A, these solutions were: DI water, DI water, DI water; for Protocol B: 8 M urea in water, DI water, DI water; for Protocol C: 8 M urea in water, phosphate buffered saline, phosphate buffered saline; for Protocol D: phosphate buffered saline, DI water, DI water; for Protocol E: 0.5 vol-% Tween 20 in water, DI water, DI water; and for Protocol F: 0.5 vol-% Tween 80 in water, DI water, DI water. Following these dialysis steps, all of the solutions were dialyzed against DI water for 48 hours before being frozen, lyophilized, and analyzed using NMR in deuterated DMSO. As a control, a portion of the solution before any dialysis was frozen, lyophilized, and analyzed by NMR to quantify the initial dopamine content in the silk solution.

Characterization of Conjugated CarboxySF-Dopamine Compositions

¹H NMR was used to confirm carboxylic acid functionalization of serine residues, quantify PEG conjugation, and confirm dopamine conjugation to the silk. All ¹H NMR spectroscopy was performed on a Bruker NMR operating at 500 MHz using TopSpin Software. Samples were run with a relaxation delay of 5 seconds, and 64 scans were averaged for each spectrum. Samples were dissolved at a concentration of 10 mg/mL in deuterated water or DMSO, as noted, and data analysis was performed using MestreC Software.

ATR-FTIR measurements were performed to study secondary structure in cast films of the materials. Scans were conducted from 4000 cm⁻¹ to 800 cm⁻¹ at a rate of 4 cm⁻¹/min, and 64 scans were averaged for each sample. To prepare samples, 40 mg/mL of the silk was dissolved in DI water and 0.7 mL of this solution was deposited onto a 1 cm diameter dish fabricated from aluminum foil. “As cast” samples were allowed to air dry at room temperature for 7 days before measuring. Induction of beta sheet was accomplished by methanol treatment. Methanol treated samples were prepared by soaking films for 24 hours in methanol (Fisher Scientific) and drying at room temperature for 48 hours before measuring. All measurements were conducted in triplicate. The background spectra were collected under the same conditions and subtracted from the scan for each sample. Fourier Self-Deconvolution (FSD) of the infrared spectra covering the Amide I region (1595˜1705 cm⁻¹) was performed with Opus 5.0 software (Bruker Optics Corp., Billerica Mass.), as described previously. Table 1 shows the average beta sheet formation of carboxylated and PEGylated silks, as determined by deconvolution of ATR-FTIR spectra acquired after treatment with methanol for 24 hours.

TABLE 1
Average Beta Sheet Formation

| | Average Beta Sheet, % |
|---|---|
| Material | (Range, %) |
| Silk (No COOH) | 39.3 |
| | (1.98) |
| Carboxy-SF (0) | 35.2 |
| | (4.70) |
| Carboxy-SF (5) | 38.7 |
| | (5.32) |
| Carboxy-SF (10) | 36.1 |
| | (3.99) |
| Carboxy-SF (23) | 38.4 |
| | (0.61) |
| Carboxy-SF (58) | 35.5 |
| | (3.67) |

Quantification of catechol functionalization to the silk protein was accomplished by Arnow's assay. In this assay, nitrate ions react with phenolic hydroxyl groups on catechols to produce NO, which causes a solution containing catechols to change color. The procedure was adopted from the literature, but a smaller scale was used to permit measurement using a microplate reader. Briefly, an aqueous solution of the silk product was prepared at a concentration of 10 mg/mL. Twenty uL of the silk solution was added to a well of a 96 well microplate. The following components were then added to the well in the prescribed order: 20 uL of 0.5 M HCl, 20 uL of a sodium nitrate/sodium molybdate solution, 20 uL of 1 M NaOH, and 20 uL distilled water. To prepare the sodium nitrate/sodium molybdate solution, 0.1 g sodium nitrate and 0.1 g of sodium molybdate were dissolved in 1 mL distilled water. Addition of the sodium nitrate/sodium molybdate solution caused a yellow color to develop in samples containing catechol, while addition of 1 M NaOH caused the solution to turn red. Absorbance at 500 nm was measured using a microplate reader. To account for changes in absorbance due to the silk protein, the appropriate silk product (CarboxySF or CarboxySF(x)) was also measured using the same procedure and the resulting absorbance was subtracted from the absorbance of the silk dopamine conjugate. Finally, quantification of conjugation was achieved using a standard curve prepared from dopamine hydrochloride.

Adhesion Studies

CarboxySF, CarboxySF(5), CarboxySF(20) and their dopamine conjugates were dissolved in water at a concentration of 100 mg/mL silk. Concentrated phosphate buffered saline (10× normal salt concentration) was added to the solution to give a solution with 2× phosphate buffer saline content. An aqueous solution of sodium periodate was prepared (75 mM), which was diluted with water to 54 mM for CarboxySF, 35 mM for CarboxySF(6), and 26 mM for CarboxySF(29). This dilution was performed so that an equal volume of periodate solution could be added to the silk solutions, thus ensuring that the silk concentration was maintained at 50 mg/mL for all compositions, while maintaining the ratio of periodate to catechol at 2.1:1. Aluminum shims (10 cm L×0.75 cm W) were placed on a flat surface, 4 μL of silk solution was added to the shim followed by 4 μL of the appropriate periodate solution. The solutions were mixed briefly using the pipet tip before overlaying a second shim (overlay was 1.0 cm L×0.75 cm W). A 13 g weight was placed on top of the shims to ensure good contact, and these were allowed to cure 24 hours at room temperature. Single lap shear testing (N=7, at minimum, for each material) was performed by mounting the shims in the tension grips of the Instron equipped with a 100 N load cell and programming the instrument to move the grips apart at a rate of 1 mm/s. The test ended when the adhesive bond ruptured, and the peak load is reported for each of these materials.

Cell Culture Studies

Films of the modified silks were prepared by casting from aqueous solutions in a 48 well tissue culture plate. At least 6 films were cast from each material. The films were allowed to dry on the benchtop for 4 days before water vapor annealing for 18 hours at room temperature to induce beta sheet formation and render the film insoluble. Films were sterilized by immersion of the plates in 70% ethanol for 45 min. The ethanol was removed from the plate, which was allowed to dry under sterile conditions. The wells were then washed 5× with sterile PBS before adding sterile medium (Dulbecco's Modified Eagles Medium (DMEM) containing 10% fetal bovine serum (FBS), and 1× antibiotic-antimycotic). The wells were washed 5× with sterile medium before soaking overnight in a 37° C. incubator with a humidified 5% CO₂ atmosphere. The films were then washed 3× additional times with sterile medium to ensure that there was no residual ethanol before hMSCs (Passage 4) were seeded on the films at 4,000 cells/cm². Cell viability and metabolic activity was measured using Alamar Blue reagent (Life Technologies), which is converted to a fluorescent product when metabolized by mammalian cells and thus is used to assay live cell function. To assay the cells, 150 uL of working solution (300 uL of Alamar Blue added to 3 mL of sterile medium) was added to each well, including acellular control wells. The plate was incubated for 2 hours at 37° C. in the 5% CO₂ incubator before 80 uL of solution was sampled from each well and read using a microplate reader operating in fluorescence mode (excitation 560 nm, emission 590 nm). The residual Alamar Blue working solution was aspirated from the wells, which were then washed 3× with sterile phosphate buffered saline before adding fresh sterile medium. Each well was measured at 1, 8, and 15 days post seeding to track cell proliferation over time.

Statistical Analysis

Univariate analysis of variance (ANOVA) will be conducted using a software package (SigmaPlot v.12.5) to determine statistical differences at the 95% confidence level (p<0.05). Multiple comparisons will be made using the Holm-Sidak method.

Results

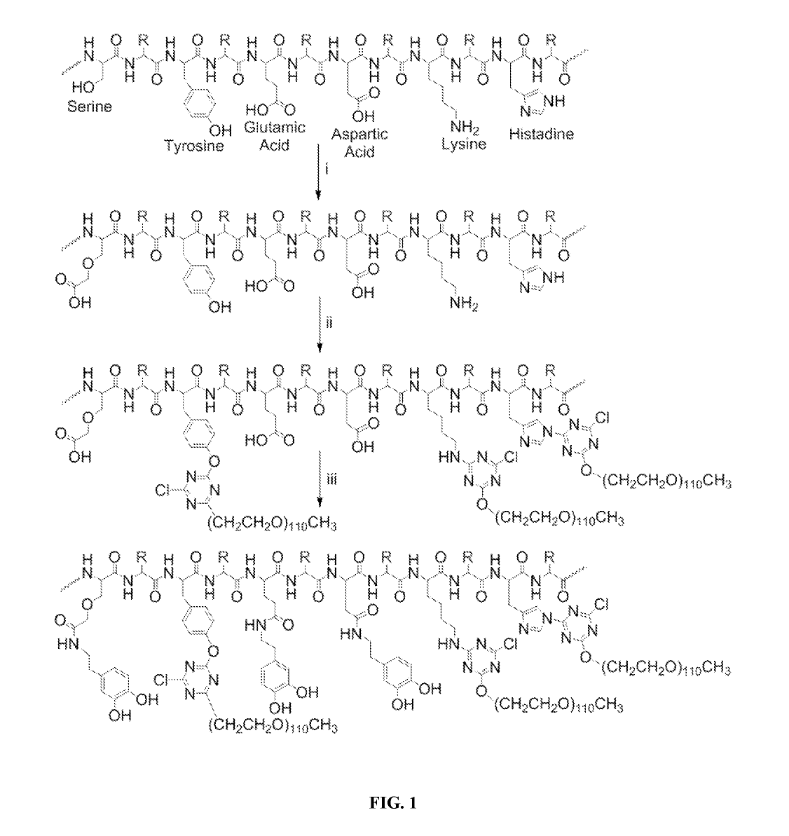

Silk fibroin was isolated from B. mori cocoons and was enriched with carboxylic acid (COOH) functional groups on serine residues as outlined in FIG. 1 Reaction i. As the heavy chain of silk fibroin contains 5,525 amino acids and serine residues account for about 11.9% of these, this reaction could theoretically increase COOH functionality from 77 groups per chain to 737 groups. Though we cannot quantify the degree of conversion of the hydroxyl groups, ¹H NMR shift of the protons on the beta carbon of serine due to change in chemical environment. Because the reaction outlined in FIG. 1 results in a very dilute product post dialysis, the CarboxySF was concentrated by air flow in a chemical fume hood for approximately 48 hours. The volume of solution removed from the tubing after the concentration step was about a third of the volume at the start of the concentration step. The solution was then lyophilized directly or conjugated with poly(ethylene glycol) (PEG) to tailor hydrophilicity.

FIG. 1 Reaction ii outlines the conjugation of PEG to silk fibroin. The amount of cyanuric chloride activated PEG fed into the reaction varied to conjugate different amounts of PEG to CarboxySF. ¹H NMR spectra of the PEGylated silks are located in Supporting Information with the methods to quantify PEG conjugation using integration of peaks. Efficiency of the conjugation, the number of chains reacted per the number of sites (tyrosine residues) that could potentially react, were found to be uniformly 34% regardless of the amount of PEG fed into the reaction.

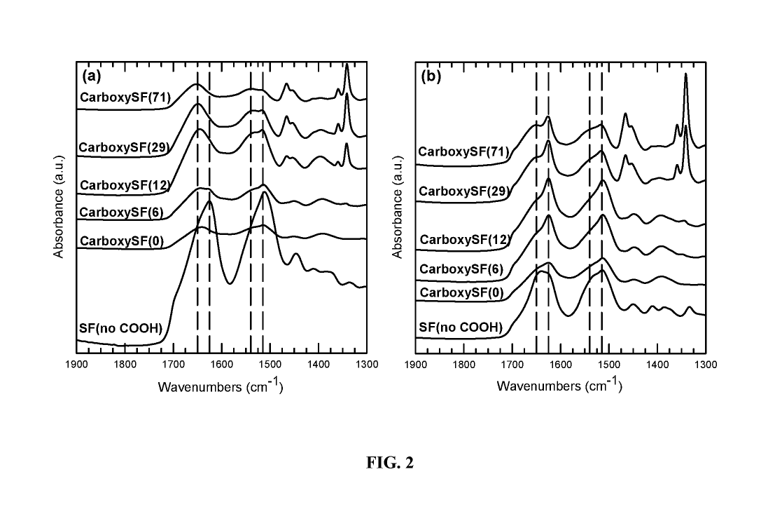

One of the goals with designing these adhesive materials was to preserve the ability of the fibroin to form beta sheet structures to afford the potential for strengthening of the material through induction of physical crosslinks. To this end, films of silk without carboxyl functionalization, CarboxySF, and the PEG conjugated carboxy silks (CarboxySF(x)) were prepared by dissolving at 40 mg/mL, casting onto an aluminum foil substrate, and air drying for 7 days. Spectra of as cast samples are shown in FIG. 2a, where both as cast CarboxySF and CarboxySF(x) adopted a random coil conformation, as evidenced by a broad peaks at 1650 cm⁻¹ and 1540 cm⁻¹. The same films were then treated with methanol for 24 hours and then dried on the bench for 48 hours before repeating the measurement (FIG. 2b). Methanol treated CarboxySF and CarboxySF(x) both showed the same changes, 1) a decrease in the peak at 1650 cm⁻¹ and the appearance of a sharp peak at 1625 cm⁻¹, and 2) a decrease in the 1540 cm⁻¹ peak and the intensification of a sharper peak at 1515 cm⁻¹. Both of these changes indicated the sample formed beta sheet secondary structures. Importantly, conjugation of PEG did not affect this transition, indicating that the PEGylated samples had the ability to be triggered to form physical crosslinks in the form of beta sheets. Fourier Self-Deconvolution (FSD) of the infrared spectra covering the Amide I region (1595˜1705 cm⁻¹) was performed as described previously to quantify the beta sheet content in the films. The secondary structure of silk fibroin was 39% beta sheet using this method (see Table 1 above). The silk derivatives were also found to form beta sheets, where the content was between 35% and 39% for CarboxySF and PEG derivatives of CarboxySF. This data suggests that the carboxylation and PEGylation reactions may slightly decrease the amount of beta sheet secondary structure, however these differences were not statistically significant in the range studied. CarboxySF, CarboxySF(6), and CarboxySF(29) were selected for study in the catechol-silk reactions to give a range of PEG substitutions, while maintaining silk as the major component in the material and the capability to form beta sheets in the material.

To ensure that dopamine was chemically coupled to silk fibroin and not absorbed, an aqueous solution of CarboxySF and dopamine was prepared, dialyzed against different solvents before being lyophilized and analyzed using ¹H NMR. Importantly, the silk did not precipitate and the solution did not turn brown (which may indicate catechol oxidation) during the dialysis for Protocols A-D. Precipitate formed during dialysis when Protocol E and F were used, thus these Protocols were not considered further as a viable option for the workup of silk-dopamine conjugates. Table 2 shows the results of this extraction study, where it was found that the wash steps in Protocol C were most effective at removing dopamine, as dopamine was not detected in the silk extracted using this method using ¹H NMR. Additionally, though no precipitation was observed in Protocols A-D, all solutions were slightly translucent except during the urea steps when the solutions were completely transparent. The change in the clarity of the solutions was attributed to protein unfolding in the presence of urea. Thus, while increasing PBS washing steps of Protocol D may result in removal of dopamine to a level achieved by Protocol C, Protocol C was selected for the final purification method of the silk-dopamine conjugates because the denaturing (unfolding) of silk proteins by urea may assist in removing unreacted dopamine.

TABLE 2
Removal of Unconjugated Dopamine

| | Dopamine Removed |
|---|---|
| Washing Method | (% of Initial) |
| Initial (No wash) | 0.00 |
| Protocol A | 93.3 |
| Protocol B | 93.2 |
| Protocol C | 100 |
| | (no dopamine detected) |
| Protocol D | 96.3 |
| Protocol E | 93.9 |
| Protocol F | 93.5 |

Table 2. Removal of unconjugated dopamine from CarboxySF (0) using different sample washing methods. After each wash, samples were lyophilized before dissolving in deuterated DMSO, measuring with ¹H NMR, and integrating the resonances from dopamine (at 2.64 and 2.91 ppm) in the resulting spectra to quantify the dopamine present in the sample.

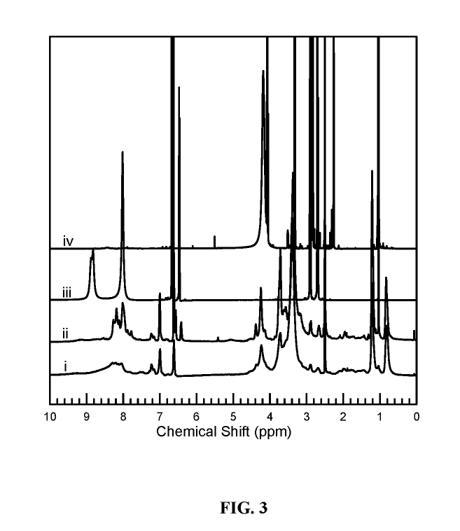

Conjugation of dopamine to CarboxySF and CarboxySF(x) is outlined in reaction iii of FIG. 1. This reaction was performed in DMSO, an organic solvent, because it was found that the pH required to react dopamine with the silk fibroin in aqueous conditions led to two issues: precipitation of the fibroin or premature oxidation of the catechol. It was necessary to avoid premature oxidation of the catechol because the oxidation reaction is involved in the adhesion and crosslinking of catechol-based adhesives. DMSO was selected because it solubilizes CarboxySF and CarboxySF(x), and because regenerated cellulose dialysis tubing is stable in DMSO, thus purification using dialysis was possible. After dialysis against DMSO, the samples were lyophilized, dissolved back into water at 50 mg/mL, washed using Protocol C with the subsequent water dialysis steps, and lyophilized to give the final product. In each case, yield of the dopamine-silk conjugate was high, 80% at minimum. FIG. 3 shows ¹H NMR spectra of CarboxySF and the CarboxySF-dopamine conjugate. Examination of the CarboxySF-dopamine (FIG. 3, trace ii) NMR spectrum shows a set of peaks between 6.45 and 6.64 ppm from the three aromatic protons of dopamine (FIG. 3, trace iii) that are not present in CarboxySF (FIG. 3, trace i). The upfield region of the spectra are more difficult to analyze due to the contributions of the fibroin to the spectra, but it is possible to discern that the 2.32 ppm peak present in COMU (FIG. 3, trace iv) is not present in CarboxySF-dopamine, indicating that the purification steps also resulted in the successful removal of COMU from the product.

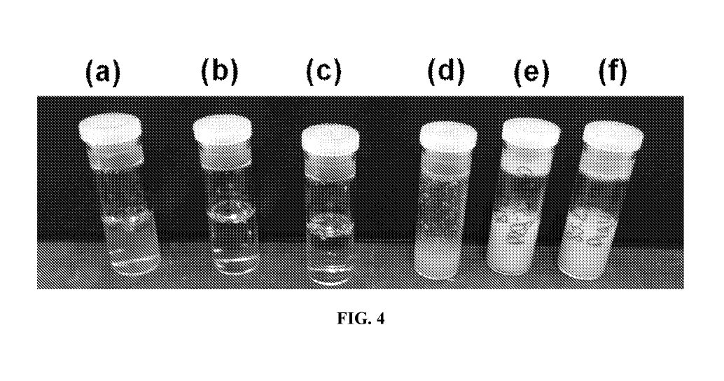

Aqueous solubility of dopamine conjugates was tested by attempting to prepare solutions at a concentration of 10 mg/mL of product. FIG. 4 shows CarboxySF, CarboxySF(20), CarboxySF(40) and their dopamine derivatives dissolved in water (pH 7.5) at a concentration of 80 mg/mL. This figure shows that all of the silks were soluble in water prior to conjugation with dopamine, however it is only the PEGylated silks that were fully soluble in water after conjugation with dopamine; CarboxySF-dopamine was only partially soluble in distilled water. Importantly, addition of PEG to the silk chains resulted in a dopamine conjugate that was completely water soluble, with as little as four PEG substitutions per molecule needed to render the dopamine conjugate soluble in water, which is highly desired for biological applications.

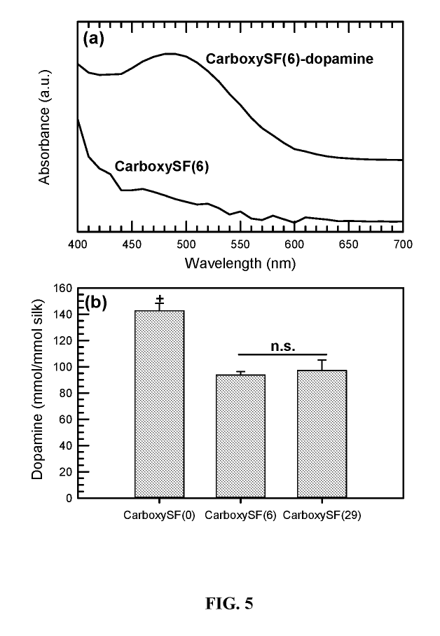

Quantification of dopamine by ¹H NMR was challenging, as silk resonances overlap many of the aliphatic resonances of dopamine and integration of the well-separated protons in the aromatic region, despite the long relaxation delays employed in these experiments. A colorimetric assay was thus used, in conjunction with a standard curve, to quantify the amount of dopamine conjugated to the fibroin. This procedure, first reported by Arnow, involves the reaction of nitrate ions in an acidic environment with phenolic alcohols to form a nitric oxide compound. FIG. 5a shows the absorbance curves from CarboxySF(6)-dopamine and CarboxySF(6). By comparison to the dopamine standard curve, the amount catechol in each sample was normalized to the silk content of each product (FIG. 5b) to show that, while conjugation was successful for all products, conjugation efficiency decreased with increased PEG functionalization. There are several possible reasons for this finding, including decreased accessibility to the reactive sites on silk due to the PEG chains, differences in the silk chain conformation in the solvent leading to decreased accessibility of reactive sites, or perhaps preparation-related reasons such as larger amounts of bound water that may poison the coupling reaction, despite the extensive drying that was applied to all samples.

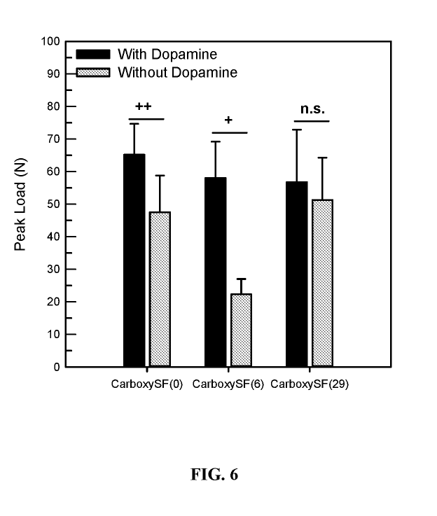

The adhesive bond of the modified silks to aluminum shims was quantified by single lap shear testing. The peak force that the adhesive was able to withstand is shown in FIG. 6, where it is shown that both the CarboxySF(0)-dopamine and CarboxySF(6)-dopamine form a stronger adhesive bond to the shims than the same material that is not functionalized with dopamine (approximate adhesive force of 55N divided by a surface area of 4.8×10⁻⁴ m², or about 115 kPa). There is no statistically significant difference between the bond strength of CarboxySF(0)-dopamine and CarboxySF(6)-dopamine, but it is noted that CarboxySF(0) has a larger peak force at break than CarboxySF(6). This indicates that silk itself, without catechol, can bond aluminum shims, a finding that is consistent with previous reports on silk hydrogels formed by electrogelation, though it is important to note that electrogelled silk hydrogel is reversible rather than permanent. While the bonding strengths between CarboxySF(0)-dopamine and CarboxySF(6)-dopamine are not statistically different, a potential advantage of the catechol coupling is the exploitation of the catechol's ability to associate with a variety of materials. Further, a small amount of PEG conjugation in CarboxySF(6)-dopamine renders the material completely soluble and therefore increases handleability compared to CarboxySF(0)-dopamine. Increasing the PEG content further increased the variability in the adhesive bond strength of the materials. The origin of this is not clear but may be for a variety of reasons, including steric or solvation effects. This Example thus emphasizes the importance of balancing solubility with dopamine functionalization to maximize the strength of the adhesion bonding.

Unique features of the silk-based dopamine adhesives are expected to arise from the structure of fibroin itself. The potential for beta sheet secondary structures to form in addition to the crosslinking between catechol residues are expected to serve as secondary crosslinks in the material that will not only strengthen the adhesive, but also hinder aqueous swelling of the polymer network. The hydrophobic nature of the silk core is also expected to reduce the degree of aqueous swelling of the silk crosslinked network and further assist in the mechanical strength of the polymer network. In this work, the tightly bonded shims did not permit adequate water penetration to conduct swelling studies, thus quantification of the swelling behavior was deferred for future studies to quantify the properties of hydrogels formed from these materials. One recognized limitation of the adhesion studies is the lack of a representative poly(ethylene glycol)-based control material that could be used to compare the adhesive bond strengths. The challenge with obtaining such a material is that the average molecular weight between crosslinks (M_{c}) of the silk-dopamine materials must be known so that a poly(ethylene glycol)-based material can be prepared with similar degrees of crosslinking, as the crosslinking will affect both the swelling and mechanical properties of the network and thus the utility of such a control. In the absence of the future mechanical studies of hydrogel networks that would permit an estimation of M_{c} from modulus and rubber elasticity theory, we thus compare the adhesive bond strengths of silk-dopamine conjugates with varying degrees of poly(ethylene glycol) substitution.

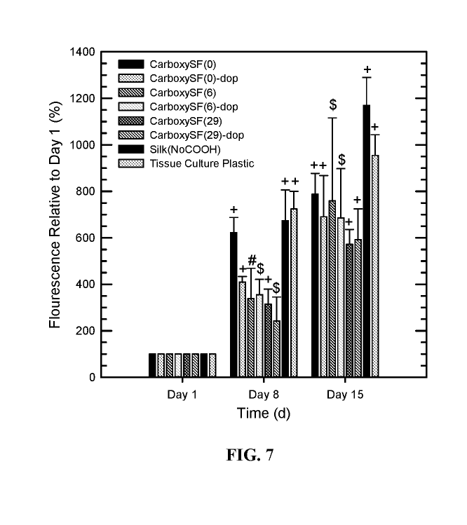

The cell compatibility of the silk and dopamine-functionalized silks was tested by seeding human mesenchymal stem cells (hMSCs). Assaying with Alamar Blue reagent permitted measurement of mitrochondrial activity in the cells, where an increase in fluorescence can be attributed to an increase in cell number and/or an increase in mitochondrial activity. Increased fluorescence was due to increased cell numbers. Cells adhered and proliferated on all silk materials tested over the course of the experiment (15 days) (FIG. 7). There were no clear trends in proliferation with the PEG functionalization and the dopamine functionalization, as the cells on all of the materials showed a statistically significant increase in fluorescence at each time point compared to their previous time point. These results thus show that all materials tested support the attachment and proliferation of hMSCs in vitro.

This Example describes the synthesis of new silk fibroin conjugates that can be triggered to crosslink by addition of an oxidant. Addition of poly(ethylene glycol) chains prior to dopamine conjugation yielded polymers with increased aqueous solubility, and this conjugation did not affect the ability of silk fibroin to form beta sheet structures. Dopamine was conjugated to the fibroin successfully. Tuning the amount of the hydrophilic PEG chains proved to be critical to maintaining solubility and the strength of the adhesive bond. All materials tested supported human mesenchymal cell attachment and proliferation throughout 15 days of in vitro culture. The additional option to induce beta sheet formation in these materials after bonding offers a path towards tunable control over the mechanics of the adhesion.

### Equivalents and Scope

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

We claim:

1. A composition comprising
silk fibroin;
at least one hydrophilic agent; and
at least one catechol donating agent, wherein the at least one hydrophilic agent and at least one catechol donating agent are conjugated to the silk fibroin.

2. The composition of claim 1, wherein the at least one hydrophilic agent is selected from the group consisting of poly(ethylene glycol), poly(glutamic acid), poly(lysine), sugars, and oligomers of sugars.

3. The composition of claim 1 or claim 2, wherein the at least one catechol donating agent is selected from the group consisting of dopamine, norepinephrine, epinephrine, and L-3,4-dihydroxyphenylalanine.

4. The composition of any one of the above claims, wherein the at least one hydrophilic agent is present in the composition in an amount at or below 10 substitutions per silk fibroin molecule.

5. The composition of claim 3, wherein the at least one hydrophilic agent is poly(ethylene glycol) and is present in the composition in an amount at or below 10 poly(ethylene glycol) chains per silk fibroin molecule.

6. The composition of claim 5, wherein the at least one hydrophilic agent is poly(ethylene glycol) and is present in the composition in an amount at or below 5 poly(ethylene glycol) chains per silk fibroin molecule.

7. The composition of any one of the above claims, wherein the silk fibroin is at least 50% crosslinked.

8. The composition of claim 7, wherein the crosslinking of the silk fibroin includes at least one of sonication, vortexing, exposure to low pH environment, methanol treatment, exposure to water vapor, exposure to shear stress, exposure to salt, exposure to elevated pressure, and exposure to an electric field.

9. The composition of any one of the above claims, wherein the composition is characterized in that, upon exposure to an aqueous environment, the composition does not swell more than 50%, relative to its original size.

10. The composition of claim 9, wherein the composition does not swell more than 20%, relative to its original size.

11. The composition of any one of the above claims, wherein the composition is characterized as having an adhesive force of at least about 20 kPa.

12. The composition of any one of the above claims, wherein the silk fibroin is functionalized through carboxylation of at least a portion of the serine groups in the silk fibroin.

13. The composition of any one of the above claims, wherein the dopamine is conjugated to the silk fibroin.

14. The composition of claim 13, wherein the conjugation is covalent bonding.

15. A method comprising
providing a silk fibroin solution;
associating the silk fibroin solution with at least one hydrophilic agent to form a solubilized silk fibroin solution; and
conjugating the solubilized silk fibroin solution with at least one catechol donating agent to form an adhesive silk fibroin composition.

16. The method of claim 15, wherein the at least one hydrophilic agent is selected from the group consisting of poly(ethylene glycol), poly(glutamic acid), poly(lysine), sugars, and oligomers of sugars.

17. The method of claim 15 or claim 16, wherein the at least one catechol donating agent is selected from the group consisting of dopamine, norepinephrine, epinephrine, and L-3,4-dihydroxyphenylalanine.

18. The method of claim 15, wherein the associating step results in not more than 10 substitutions being associated per silk fibroin molecule.

19. The method of claim 18, wherein the associating step results in not more than 5 chains being associated per silk fibroin molecule.

20. The method of claim 17, wherein the at least one hydrophilic agent is poly(ethylene glycol) and is present in the composition in an amount at or below 10 poly(ethylene glycol) chains per silk fibroin molecule.

21. The method of claim 20, wherein the at least one hydrophilic agent is poly(ethylene glycol) and is present in the composition in an amount at or below 5 poly(ethylene glycol) chains per silk fibroin molecule.

22. The method of any one of claims 15-21, wherein the silk fibroin in the silk fibroin solution is functionalized through carboxylation of at least a portion of the serine groups in the silk fibroin prior to the associating step.

23. The method of any one of claims 15-22, wherein the adhesive silk fibroin composition is characterized as having an adhesive force of at least about 20 kPa.

24. The method of any one of 15-23, wherein the adhesive silk fibroin composition does not swell more than 50%, relative to its original size, upon exposure to an aqueous environment.

25. The method of any one of claims 15-24, wherein the silk fibroin solution is lyophilized prior to the associating step.

26. The method of any one of claims 15-25, wherein the silk fibroin solution is lyophilized prior to the conjugating step.

27. The method of any one of claims 15-26, further comprising crosslinking at least a portion of the silk fibroin in the adhesive silk fibroin composition.

28. The method of claim 27, wherein the crosslinking occurs after the associating step.

29. The method of claim 27, wherein the crosslinking occurs after the conjugating step.

30. The method of any one of claims 27-29, wherein the crosslinking of the silk fibroin includes at least one of sonication, vortexing, exposure to low pH environment, methanol treatment, exposure to water vapor, exposure to shear stress, exposure to salt, exposure to elevated pressure, and exposure to an electric field.
